(19) United States
(12) Patent Application Publication
(10) Pub. No.: US 2017/0332979 A1
(43) Pub. Date: Nov. 23, 2017

(54) TECHNOLOGIES FOR SYNCHRONIZING PHYSIOLOGICAL FUNCTIONS

(71) Applicant: Intel Corporation, Santa Clara, CA (US)

(72) Inventors: Ramune Nagisetty, Portland, OR (US); Daria A. Loi, Portland, OR (US); Giuseppe Raffa, Portland, OR (US); Bradley A. Jackson, Hillsboro, OR (US)

(21) Appl. No.: 15/161,362

(22) Filed: May 23, 2016

(51) Int. Cl. A61B 5/00; A61B 5/0205

(52) U.S. Cl. CPC A61B 5/7282; A61B 5/7203; A61B 5/742; A61B 5/7225; A61B 5/002; A61B 5/6801; A61B 5/0205; A61B 5/0022; A61B 2560/0242

(57) ABSTRACT

Technologies for synchronizing physiological functions of people include a wearable compute device. The wearable compute device is to determine physiological rate data of a user of the wearable device based on sensor data produced by at least one physiological sensor included in the wearable compute device, determine a difference between the determined physiological rate data of the user and reference physiological rate data, generate a perceptible signal that is representative of the determined difference, and convey the perceptible signal to the user with at least one signal conveyor device included in the wearable compute device, to facilitate synchronization of the physiological functions of the user with the reference physiological rate data. Other embodiments are described.

### BACKGROUND

Some physiological functions, such as heart rates, respiration rates, and heart rate variability (also known as respiratory sinus arrhythmia) have been shown to be more synchronized among people in a social relationship when those people are in close physical proximity to each other. It is believed that synchronization of these physiological functions can be beneficial both physically and mentally. For example, synchronization of the physiological functions may improve cardiovascular health and strengthen the social relationship between the people involved in the relationship. When people are not in close proximity with each other, such as when they are in a long-distance relationship, their physiological functions are unlikely to be synchronized.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a system for facilitating synchronization of physiological functions of people;

FIG. 2 is a simplified block diagram of at least one embodiment of a wearable compute device of the system of FIG. 1;

FIG. 3 is a simplified block diagram of at least one embodiment of an environment that may be established by a wearable compute device of FIGS. 1 and 2; and

FIGS. 4-6 are a simplified flow diagram of at least one embodiment of a method for facilitating synchronization of physiological functions that may be performed by the wearable compute device of FIGS. 1 and 2.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

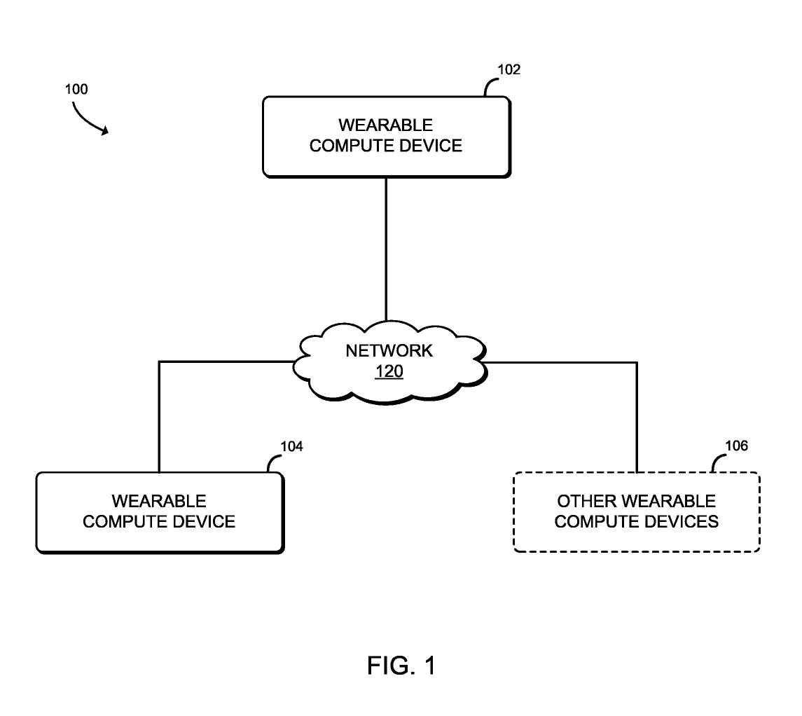

Referring now to FIG. 1, in an illustrative embodiment, a system for facilitating synchronization of physiological functions of people includes a set of wearable compute devices 100 in communication through a network. The wearable compute devices 100 include a wearable compute device 102, another wearable compute device 104, and optionally, other wearable compute devices 106. In operation, the illustrative wearable compute devices 100 communicate data with each other regarding physiological functions of their respective users, and provide stimuli to their users based on the communicated data, to assist in helping the users feel as if they are in close proximity to each other when they may be separated by a significant distance. By doing so, the illustrative wearable compute devices 100 facilitate synchronizing the physiological functions of their respective users. In the illustrative embodiment, each wearable compute device 100 is configured to communicate data regarding respiration rates, heart beat rates (“heart rates”), and/or heart rate variability. Further, each illustrative wearable compute device 100 is configured to receive the transmitted physiological rate data and perform actions to aid in synchronizing the wearer's physiological rates (e.g., respiration rate, heart rate, heart rate variability, etc.) with the received rates. The wearable compute devices 100 may aid in synchronizing these physiological rates by providing stimuli to their wearers, such as haptic signals, visual signals, audible signals, and/or thermal signals that represent the received physiological rate and/or the difference between the wearer's physiological rate and the received physiologic rate data. Additionally, the wearable compute devices 100 may be configured to provide reminders, such as images, videos, and/or sounds, to their wearers of times when their physiological functions were more synchronized with those of the other wearers. By providing these stimuli, the physiological functions of each wearer may become more synchronized and provide a feeling of closeness.

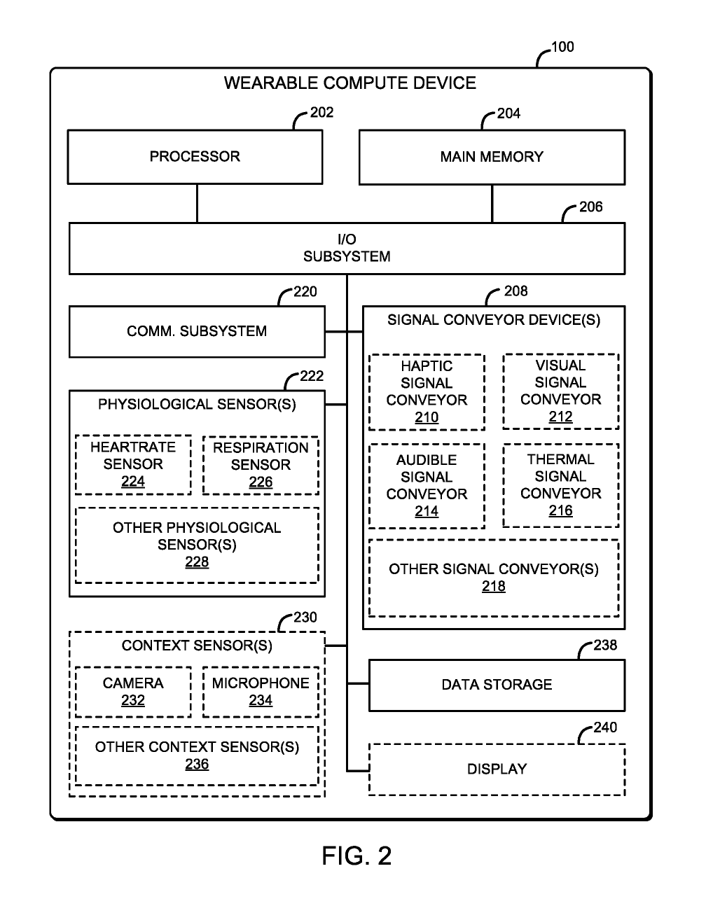

Referring now to FIG. 2, each wearable compute device 100 may be embodied as any type of compute device capable of performing the functions described herein. For example, in some embodiments, each wearable compute device 100 may be embodied as or incorporated into, without limitation, a wearable article of clothing or jewelry, such as a wristband, a head band, a belt, an anklet, a necklace, gloves, eye glasses, a smart phone, a tablet, and/or any other computing device capable of performing functions to synchronize the physiological functions of their wearers, as described herein. As shown in FIG. 2, the illustrative wearable compute device 100 includes a processor 202, a main memory 204, an input/output subsystem 206, one or more signal conveyor devices 208, a communication subsystem 220, one or more physiological sensors 222, and optionally, one or more context sensors 230. Of course, the wearable compute device 100 may include other or additional components, such as those commonly found in a computer (e.g., data storage, one or more displays, etc.), in other embodiments. Additionally, in some embodiments, one or more of the illustrative components may be incorporated in, or otherwise from a portion of, another component. For example, the memory 204, or portions thereof, may be incorporated in the processor 202 in some embodiments.

The processor 202 may be embodied as any type of processor capable of performing the functions described herein. For example, the processor may be embodied as a single or multi-core processor(s) having one or more processor cores, a digital signal processor, a microcontroller, or other processor or processing/controlling circuit. Similarly, the main memory 204 may be embodied as any type of volatile or non-volatile memory or data storage capable of performing the functions described herein. In operation, the main memory 204 may store various data and software used during operation of the wearable compute device 100 such as determined physiological rate data from a wearer of the wearable compute device 100, reference physiological rate data to which the wearable compute device 100 may attempt to synchronize the wearer's physiological functions, threshold data indicative of various thresholds used in determining degrees of synchronization between wearers, contextual data indicative of environmental conditions or other surrounding circumstances of the wearer, operating systems, applications, programs, libraries, and drivers. The main memory 204 is communicatively coupled to the processor 202 via the I/O subsystem 206. Of course, in other embodiments (e.g., those in which the processor 202 includes a memory controller), the main memory 204 may be directly communicatively coupled to the processor 202.

The I/O subsystem 206 may be embodied as circuitry and/or components to facilitate input/output operations with the processor 202, the main memory 204, and other components of the wearable compute device 100. For example, the I/O subsystem 206 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 206 may form a portion of a system-on-a-chip (SoC) and be incorporated, along with the processor 202, the memory 204, and other components of the wearable compute device 100, on a single integrated circuit chip.

The illustrative wearable compute device 100 additionally includes the signal conveyor devices 208, each of which is configured to provide perceptible signals to the wearer of the wearable compute device 100. In the illustrative embodiment, the wearable compute device 100 includes at least one of a haptic signal conveyor 210, a visual signal conveyor 212, an audible signal conveyor 214, a thermal signal conveyor 216, or other signal conveyors 218. Of course, in other embodiments, the wearable compute device 100 may include additional or different signal conveyor devices 208. The haptic signal conveyor 210 may be embodied as any type of device capable of providing pulses, vibrations, pressure, or other stimuli to be felt by the wearer of the wearable compute device 100. For example, the haptic signal conveyor 210 may be embodied as an eccentric rotating mass, a linear resonant actuator, piezoelectric transducers, an air bladder configured to selectively inflate or deflate, magnets, or shape memory alloys such as nitinol springs, to tighten and loosen the wearable compute device 100 when worn around a wrist, or otherwise provide tactile sensations to the wearer. In the illustrative embodiment, the haptic signals are indicative of physiological function rates or differences between physiological function rates.

The illustrative visual signal conveyor 212 may be embodied as any type of device capable of providing visual stimuli to a wearer of the wearable compute device 100. The visual signals may be lights or graphical representations of heart rates, respiration rates, or other physiological function rates of the wearer or a person with whom the wearer is to synchronize his or her physiological function rates, or a representation of a difference between the two sets of rates. As described in more detail herein, the visual signal conveyor may also be configured to provide images or video associated with a context in which the physiological functions of the wearer and another person were determined to be synchronized. Accordingly, the visual signal conveyor 212 may be embodied as, or otherwise use, any suitable display technology including, for example, a light emitting device, such as a light emitting diode (LED), or a graphical display such a liquid crystal display (LCD), a light emitting diode (LED) display, a cathode ray tube (CRT) display, a plasma display, and/or other display usable in a compute device.

The illustrative audible signal conveyor 214 may be embodied as any type of device capable of conveying audible stimuli to a wearer of the wearable compute device 100 indicative of physiological function rates or differences between physiological function rates. Additionally, in the illustrative embodiment, the audible signal conveyor is further configured to provide audible signals, such as environmental sounds, speech, or other audio associated with a previous context in which the physiological functions of the wearer and another person were determined to be synchronized. For example, the audible signal conveyor 214 may be embodied as a speaker or other device capable of emitting sound, including bone conduction.

The illustrative thermal signal conveyor 216 may be embodied as any type of device capable of providing thermal stimuli to a wearer of the wearable compute device 100 indicative of physiological function rates or differences between physiological function rates. For example, the thermal signal conveyor 216 may increase in temperature to indicate that the wearable compute device 100 has determined that the physiological functions of the wearer and another person have become synchronized. In other embodiments, the thermal signal conveyor 216 may decrease in temperature to indicate that the physiological functions of the wearer and the other person have become synchronized. In some embodiments, the thermal signal generator 216 may selectively increase or decrease its temperature to indicate other information regarding the wearer's and the other person's physiological function rates. The thermal signal conveyor may be embodied as an electrically resistive material that increases in temperature when an electrical current passes through it, a heat exchanger device to transfer heat away from the portion of the wearer's body in contact with the thermal signal conveyor, or may include other components suitable to perform the functions described above. As referenced above, the signal conveyor devices 208 may include other signal conveyors 218 capable of providing perceptible signals to the wearer regarding the physiological functions of the wearer and of another person (i.e., a wearer of another wearable compute device 100).

The illustrative wearable compute device 100 additionally includes the communication subsystem 220. The communication subsystem 220 may be embodied as one or more devices and/or circuitry capable of enabling communications with one or more other wearable compute devices 100 over a network. The communication subsystem 220 may be configured to use any suitable communication protocol to communicate with other devices including, for example, wireless data communication protocols, cellular communication protocols, and/or wired communication protocols.

The illustrative wearable compute device 100 additionally includes the physiological sensor(s) 222. Each physiological sensor 222 may be embodied as any type of device capable of sensing a physiological characteristic of a user. The illustrative physiological sensors 222 include a heartrate sensor 224, a respiration sensor 226, and/or other physiological sensors 228 capable of sensing other physiological functions of the wearer, such as changes in body temperature. The illustrative heartrate sensor 224 may be embodied as any device capable of detecting electrical signals, changes in blood absorption of light (i.e., infrared light), or changes in blood pressure, associated with beats of the heart. The respiration sensor 226 may be embodied as any device capable of detecting breathing rates of the wearer, and may include a pressure sensor to detect expansion and contraction of the torso, a microphone to capture the sounds of air moving through the lungs, or other devices capable of detecting respiration rates of the wearer.

The illustrative wearable compute device 100 additionally includes one or more context sensors 230 capable of detecting aspects of the environment or circumstances surrounding the wearer of the wearable compute device 100. The context sensors 230 may be embodied as, or otherwise include, a camera 232, a microphone 234, or other sensors 236, such as a thermometer or a location sensor such as a global positioning system (GPS) sensor. As described in more detail herein, the wearable compute device 100 may determine at various times that the physiological functions of the wearer and other person are similar enough to be deemed synchronized and store information about the context of the wearer using the context sensors 230. The context may be embodied as, for example, an image of the wearer and the other person, a video of them, or audio of speech or sounds associated with the time when the physiological functions were determined to be synchronized.

The wearable compute device 100 may additionally include a data storage device 238, which may be embodied as any type of device or devices configured for short-term or long-term storage of data such as, for example, memory devices and circuits, memory cards, hard disk drives, solid-state drives, or other data storage devices. The data storage device 238 may store data and software used during operation of the wearable compute device 100 such as determined physiological rate data from a wearer of the wearable compute device 100, reference physiological rate data to which the wearable compute device 100 may attempt to synchronize the wearer's physiological functions, threshold data indicative of various thresholds used in determining whether synchronization has been established between wearers, contextual data indicative of environmental conditions or other surrounding circumstances of the wearer, operating systems, applications, programs, libraries, and drivers, as described in more detail herein.

The wearable compute device 100 may additionally include a display 240, which may be embodied as any type of display device on which information may be displayed to a wearer of the wearable compute device 100. The display 240 may be embodied as, or otherwise use, any suitable display technology including, for example, a liquid crystal display (LCD), a light emitting diode (LED) display, a cathode ray tube (CRT) display, a plasma display, and/or other display usable in a compute device. The display 240 may include a touchscreen sensor that uses any suitable touchscreen input technology to detect the user's tactile selection of information displayed on the display including, but not limited to, resistive touchscreen sensors, capacitive touchscreen sensors, surface acoustic wave (SAW) touchscreen sensors, infrared touchscreen sensors, optical imaging touchscreen sensors, acoustic touchscreen sensors, and/or other type of touchscreen sensors. In some embodiments, the visual signal conveyor 212 and the display 240 may be the same component while in other embodiments, they are distinct components.

Referring back to FIG. 1, the network 120 may be embodied as any number of various wireless or wired networks. For example, the network 120 may be embodied as, or otherwise include, a publicly-accessible, global network such as the Internet, a cellular network, a wireless or wired wide area network (WAN), or a wireless local area network (LAN). As such, the network 120 may include any number of additional devices, such as additional computers, routers, and switches, to facilitate communications among the devices of the system.

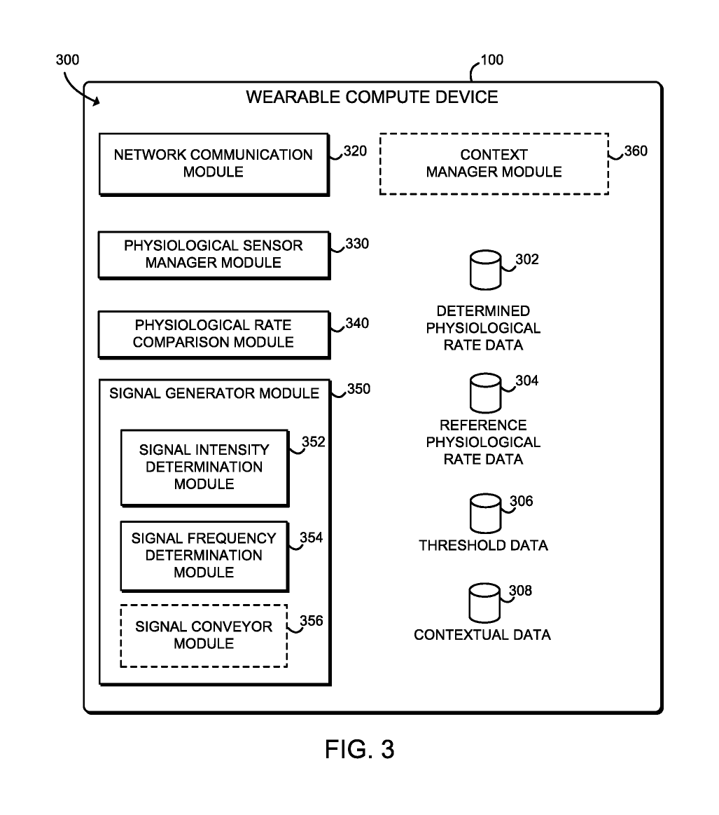

Referring now to FIG. 3, in the illustrative embodiment, each wearable compute device 100 may establish an environment 300 during operation. The illustrative environment 300 includes a network communication module 320, a physiological sensor manager module 330, a physiological rate comparison module 340, a signal generator module 350, and a context manager module 360. Each of the modules, logic, and other components of the environment 300 may be embodied as hardware, firmware, software, or a combination thereof. As such, in some embodiments, one or more of the modules of the environment 300 may be embodied as circuitry or collection of electrical devices (e.g., network communication circuitry 320, physiological sensor manager circuitry 330, physiological rate comparison circuitry 340, signal generator circuitry 350, context manager circuitry 360, etc.). It should be appreciated that, in such embodiments, one or more of the network communication circuitry 320, physiological sensor manager circuitry 330, physiological rate comparison circuitry 340, signal generator circuitry 350, and context manager circuitry 360 may form a portion of one or more of the processor 202, signal conveyor devices 208, physiological sensors 222, context sensors 230, and/or other components of the wearable compute device 100. Additionally, in some embodiments, one or more of the illustrative modules may form a portion of another module and/or one or more of the illustrative modules may be independent of one another. Further, in some embodiments, one or more of the modules of the environment 300 may be embodied as virtualized hardware components or emulated architecture, which may be established and maintained by the processor 202 or other components of the wearable compute device 100.

In the illustrative environment 300, the wearable compute device 100 also includes determined physiological rate data 302 generated or produced using the physiological sensors 222, reference physiological rate data 304 received by the wearable compute device 100 that indicates physiological rates of another wearer of another wearable compute device 100 and to which the wearable compute device 100 may attempt to synchronize the wearer's physiological functions, threshold data 306 indicative of thresholds to be used in determining whether physiological rates are synchronized between wearers, and contextual data 308 indicative of environmental conditions or other surrounding circumstances (e.g., images, audio, and/or video) of the wearer. The determined physiological rate data 302, the reference physiological rate data 304, the threshold data 306, and the contextual data 308 may be accessed by the various modules and/or sub-modules of the wearable compute device 100. It should be appreciated that the wearable compute device 100 may include other components, sub-components, modules, sub-modules, and/or devices commonly found in a compute device, which are not illustrated in FIG. 3 for clarity of the description.

The network communication module 320, which may be embodied as hardware, firmware, software, virtualized hardware, emulated architecture, and/or a combination thereof as discussed above, is configured to manage inbound and outbound network communications to and from the wearable compute device 100, respectively. For example, the network communication module 320 is configured to transmit determined physiological rate data 302 (e.g., heart rate data, respiration rate data, etc.), and receive reference physiological data (e.g., heart rate data, respiration rate data, etc. of at least one wearer of another wearable compute device 100). The network communication module 320 may further be configured to pair with one or more other wearable compute devices 100 prior to communicating with the wearable compute devices 100. Further, the network communication module 320 may be configured to determine a latency in communications with the other wearable compute devices(s) 100. In some embodiments, at least a portion of the functionality of the network communication module 320 may be performed by the communication subsystem 220 of FIG. 2.

The physiological sensor manager module 330, which may be embodied as hardware, firmware, software, virtualized hardware, emulated architecture, and/or a combination thereof as discussed above, is configured to determine physiological rate data 302 of the wearer based on sensor data produced by the physiological sensors 222. The illustrative physiological sensor manager module 330 may be configured to receive sensor data indicative of heart beats or inhalations and/or exhalations over a predefined time period, such as ten seconds, and extrapolate the sensor data from that time period to a longer time period, such as a minute. The physiological sensor manager module 330 may also be configured to determine average rates of various physiological functions (e.g., breathing, heart beats, etc.). Further, the physiological sensor manager module 330 may be configured to determine a variability in the heart rate and/or the respiration rates. Moreover, the physiological sensor manager module 330 may be configured to apply one or more filters or otherwise refine sensor data received from the physiological sensors 222 to remove noise, such as by applying a bandpass filter to the sensor data.

The illustrative physiological rate comparison module 340, which may be embodied as hardware, firmware, software, virtualized hardware, emulated architecture, and/or a combination thereof as discussed above, is configured to compare physiological rates indicated in the determined physiological rate data 302 to physiological rates represented in the reference physiological rate data 304. The illustrative physiological rate comparison module 340 is configured to determine a difference between the physiological rates of the respective sets of data, such as a difference in frequency, phase (i.e., a time offset), and/or amplitude. The physiological rate comparison module 340 may be configured to adjust the reference physiological rate data 304 by a latency determined by the network communication module 320 prior to performing the comparison. The physiological rate comparison module 340 may additionally be configured to identify an anomaly in the determined physiological rate data 302 and remove the anomaly from the determined physiological rate data 302 prior to performing the comparison with the reference physiological rate data 304. For example, the physiological rate comparison module 340 may be configured to determine whether the wearer of the wearable compute device 100 is presently engaged in a strenuous activity, such as running, swimming, or other exercise, based on an increase in the physiological rates of the wearer that satisfies a predefined threshold change (i.e., a 25% increase in heart rate or respiration rate) and delete or ignore information pertaining to this time period when comparing the determined physiological rate data 302 to the reference physiological rate data 304.

The illustrative signal generator module 350, which may be embodied as hardware, firmware, software, virtualized hardware, emulated architecture, and/or a combination thereof as discussed above, is configured to generate a perceptible signal that is representative of the determined difference between the determined physiological rate data 302 and the reference physiological rate data 304 and convey the perceptible signal to the user with at least one of the signal conveyor devices 208 to facilitate synchronization of the physiological functions of the user with the wearer(s) of the other wearable compute device(s) 100. To do so, the illustrative signal generator module 350 includes a signal intensity determination module 352, a signal frequency determination module 354, and a signal conveyor module 356.

The signal intensity determination module 352 is configured to determine an intensity of the perceptible signal to be conveyed to the wearer of the wearable compute device 100. In the illustrative embodiment, the signal intensity determination module 352 is configured to selectively increase or decrease the intensity based on the determined difference between the determined physiological rate data 302 and the reference physiological rate data 304. As an example, the signal intensity determination module 352 may be configured to increase the intensity from a baseline level in response to an increase in the determined difference, and to decrease the intensity in response to a decrease in the determined difference decrease. The intensity may be embodied differently depending on the type of signal conveyor device 208 used to output the signal. For a haptic signal, the intensity may be a pressure or an amplitude of vibration. For a visual signal, the intensity may be a brightness, color, graphical image, or pattern. For an audible signal, the intensity may be a volume, and for a thermal signal, the intensity may be a temperature.

The illustrative signal frequency determination module 354 is configured to determine a frequency of the perceptible signal to be conveyed to the wearer of the wearable compute device 100. In the illustrative embodiment, the determined frequency is the frequency of a reference physiological function (e.g., a heart rate or respiration rate) represented in the reference physiological rate data 304. The frequency may be embodied in various forms, depending on the signal conveyor device 208 used to output the signal. For example, the frequency may be a vibration rate, a color or rate or rate of blinking, a pitch or rate of beeping, etc.

The illustrative signal conveyor module 356 is configured to transmit an electrical signal to the corresponding signal conveyor device(s) 208 to produce a perceptible signal in accordance with the intensity and frequency determined by the signal intensity determination module 352 and the signal frequency determination module 354, respectively. The electrical signal may be a data signal, in which the signal indicates the determined intensity and frequency, or may be a power signal, in which the signal provides the electrical power to produce the signal at the determined intensity and frequency.

It should be appreciated that each of the signal intensity determination module 352, the signal frequency determination module 354, and the signal conveyor module 356 may be separately embodied as hardware, firmware, software, virtualized hardware, emulated architecture, and/or a combination thereof. For example, the signal intensity determination module 352 may be embodied as a hardware component, while the signal frequency determination module 354 and the signal conveyor module 356 are embodied as virtualized hardware components or as some other combination of hardware, firmware, software, virtualized hardware, emulated architecture, and/or a combination thereof.

The illustrative context manager module 360, which may be embodied as hardware, firmware, software, virtualized hardware, emulated architecture, and/or a combination thereof as discussed above, is configured to determine contextual data indicative of a present context of the wearer. In the illustrative embodiment, the context manager module 360 is configured to store time stamps in association with the contextual data, data indicative of the determined difference between the determined physiological rate data 302 and the reference physiological rate data 304 of at least one other person, an indication of an identity of the other person or of the other person's wearable compute device 100, and an indication of whether the physiological rate comparison module 340 determined that the compared physiological rates of the wearer and the other person were synchronized. The context manager module 360 is illustratively configured to determine the contextual data using one or more of the context sensors 230, such as by storing image or video data of an environment of the wearer and the other person (e.g., an image or video of the wearer and the other person walking on a beach) or audio data (e.g., a recording of a conversation between the wearer and the other person). Further, the illustrative context manager module 360 is configured to present contextual data 308 associated with a time when the physiological rates were determined to be synchronized. The illustrative context manager module 360 may be configured to present the contextual data 308 to the wearer in response to a request from the wearer (e.g., a spoken request, a request through a user interface displayed by the display 240, etc.) and/or in response to the physiological rate comparison module 340 determining that the difference between a physiological rate of the wearer and of other person presently satisfies a predefined threshold difference. By presenting the wearer with such contextual data, the wearable compute device 100 may prompt the wearer to experience a feeling of closeness with the other person and begin to synchronize his or her physiological functions with those of the other person.

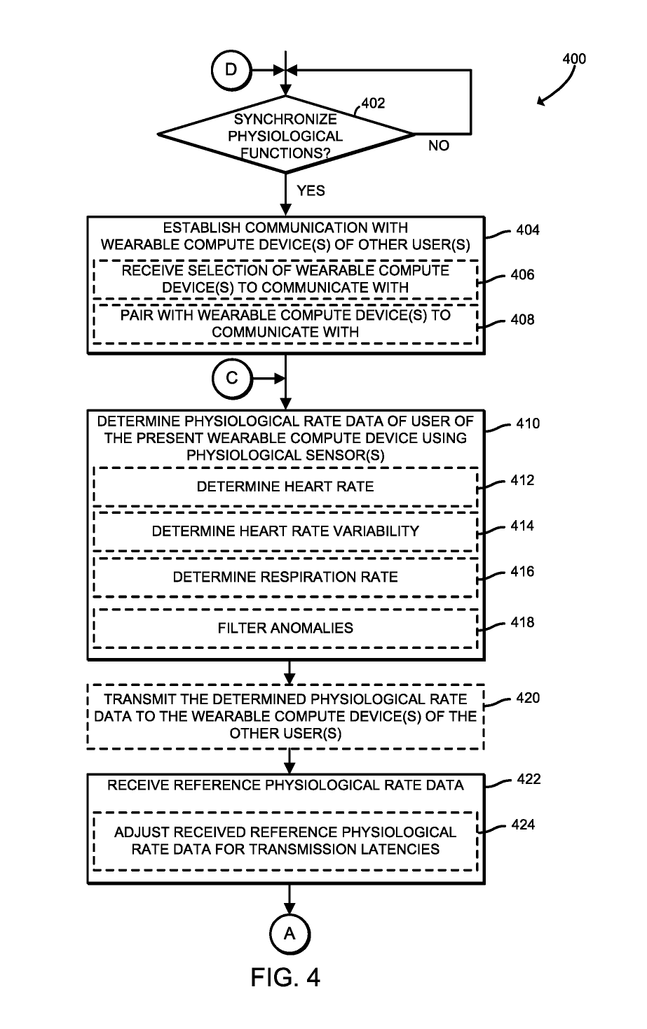

Referring now to FIG. 4, in use, the wearable compute device 100 may execute a method 400 for facilitating synchronization of physiological functions between a wearer of the wearable compute device 100 (e.g., the wearable compute device 102) and a wearer of another wearable compute device (e.g., the wearable compute device 104). The method 400 begins with block 402, in which one of the wearable compute devices 100, such as the wearable compute device 102, determines whether to facilitate synchronization of physiological functions with those of at least one other person wearing another wearable compute device 100, such as a wearer of the wearable compute device 104. In the illustrative embodiment, the wearable compute device 100 determines to facilitate synchronization of physiological functions in response to a user request provided through a graphical user interface (not shown) presented by the wearable compute device 100. In other embodiments, the wearable compute device 100 may determine to facilitate synchronization of physiological functions in response to detecting the presence, such as a wireless data signal, of one or more other wearable compute devices 104, 106. Regardless, if the wearable compute device 100 determines to facilitate synchronization of physiological functions, the method 400 advances to block 404 in which the wearable compute device 100 establishes communication with the wearable compute device(s) 100 of other user(s). In doing so, as indicated in block 406, the wearable compute device 100 may receive a selection of one or more of the other wearable compute devices 100 to communicate with, such as through a graphical user interface (not shown) presented by the wearable compute device 100. As indicated in block 408, the wearable compute device 100 may pair with the other wearable compute device(s) 100, such as by exchanging device identifiers (e.g., names, serial numbers, media access control (MAC) addresses, etc.), communication settings, and, in some embodiments passwords, PINs, or other authorization credentials.

In block 410, the wearable compute device 100 determines the physiological rate data 302 of the user (i.e., wearer) of the present wearable compute device 100 using the physiological sensor(s) 222. In doing so, the wearable compute device 100 may determine a heart rate of the user as indicated in block 412. As indicated in block 414, the wearable compute device 100 may determine a heart rate variability of the user. Additionally or alternatively, the wearable compute device 100 may determine a respiration rate of the user, as indicated in block 416. In determining the above rates, the wearable compute device 100 may receive sensor data produced by the respective physiological sensors 222 over a time period, such as ten seconds, and extrapolate the sensor data to a longer time period, such as a minute. Further, the wearable compute device 100 may determine an average physiological rate over several iterations of a predefined time period, such as an average heart rate over several minutes. As indicated in block 418, the wearable compute device 100 may filter anomalies from the determined physiological rate data 302 such as by applying a bandpass filter to eliminate noise in frequency bands that are not associated with the physiological functions to be monitored and/or removing physiological rate data associated with exercise or other activities that temporarily change the rate of the physiological functions by a threshold amount.

In the illustrative embodiment, in block 420, the wearable compute device 100 transmits the determined physiological rate data 302 to the wearable compute device(s) 100 of the other user(s) with which the wearable compute device 100 established communication in block 404. Subsequently, in block 422, the wearable compute device 100 receives reference physiological rate data 304. In the illustrative embodiment, the wearable compute device 100 receives the reference physiological rate data 304 from the wearable compute device(s) of the other user(s). In receiving the reference physiological rate data, the wearable compute device 100 may determine latencies in the communications and adjust the received reference physiological rate data 304 based on the transmission latencies, as indicated in block 424. This may be beneficial in enabling the wearable compute device 100 to more accurately determine a degree to which the physiological rates of the wearer differ from those of the other user(s). In some embodiments, the wearable compute device 100 receives or obtains predefined reference physiological rate data that is not necessarily indicative of physiological function rates of any of the users. Rather, the predefined reference physiological rate data may be representative of a target set of physiological function rates that the users wish to synchronize their physiological functions with (e.g., a target heart rate during an exercise class, etc.).

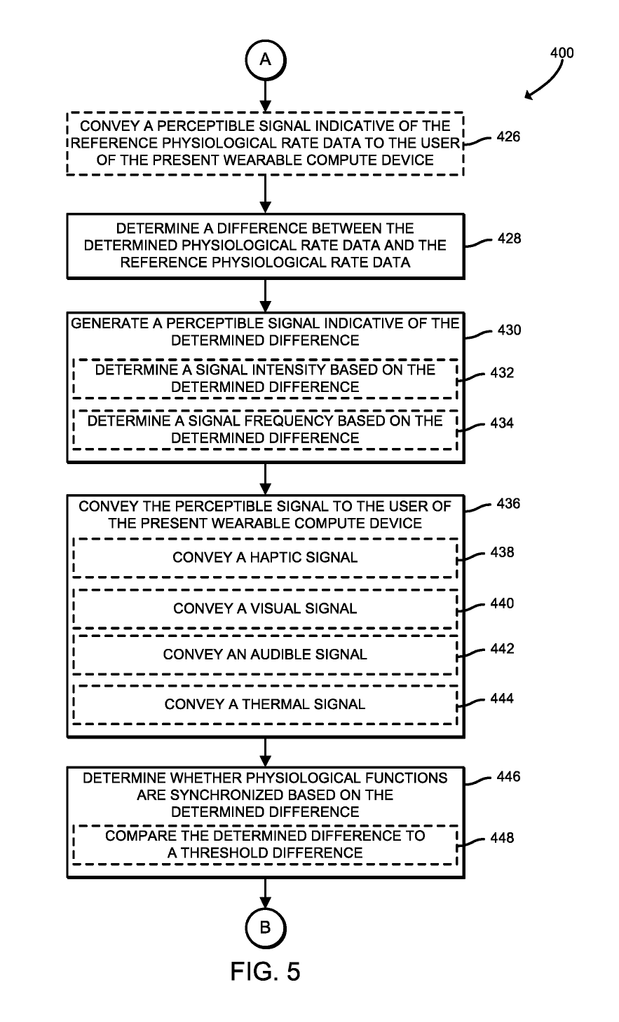

After the wearable compute device 100 receives the reference physiological rate data 304 in block 424, the method 300 advances to block 426 of FIG. 5 in some embodiments. In block 426, the illustrative wearable compute device 100 may convey a perceptible signal indicative of the reference physiological rate data 304 to the user of the present wearable compute device 100. In block 428, the wearable compute device 100 determines a difference between the determined physiological rate data 302 and the reference physiological rate data 304. In doing so, the wearable compute device 100 may determine differences of various aspects, including phases, amplitudes, and/or frequencies, of the corresponding physiological rates. In some embodiments, the wearable compute device 100 may consolidate the differences into a total value that is representative of the differences in the various aspects. In block 430, the wearable compute device 100 generates a perceptible signal indicative of the determined difference between the determined physiological rate data 302 and the reference physiological rate data 304. In doing so, the wearable compute device 100 may determine a signal intensity based on the determined difference, as indicated in block 432. For example, the wearable compute device 100 may set the intensity (i.e., volume, brightness, etc.) of the signal higher the more out of synchronization (i.e., the greater the difference) the compared physiological rates are, and lower the intensity the more synchronized the compared physiological rates are. In other embodiments, the wearable compute device 100 may operate in the opposite manner, increasing the intensity as the determined difference decreases and decreasing the intensity as the determined difference increases. As indicated in block 434, the wearable compute device 100 may determine a signal frequency (i.e., pitch, color, rate of blinking, rate of beeping, rate of vibration, etc.) based on the determined difference. In other embodiments, the frequency may be the rate of a physiological function represented in the reference physiological rate data 304, such as the heart rate or respiration rate of a person to whom the wearer of the wearable compute device 100 is to synchronize their physiological functions.

In block 436, the wearable compute device 100 conveys the perceptible signal to the user of the present wearable compute device 100. As described above, the perceptible signal may be embodied in various forms depending on the signal conveyor device(s) 208 used to produce the perceptible signal. As indicated in block 438, the wearable compute device 100 may convey a haptic signal to the wearer. As described above, the haptic signal may be a vibration, a pulse, a tightening or loosening of the wearable compute device around a portion of the wearer's body, or other tactile sensation. Additionally or alternatively, as indicated in block 440, the wearable compute device 100 may convey a visual signal to the wearer, such as a blinking and/or colored light, a graphical display of the determined difference such as a numeric value, an icon, or other visual representation of the determined difference. As indicated in block 442, the wearable compute device 100 may convey an audible signal, such as a breathing sound, a heartbeat sound, a beeping sound, a tone, or other audible representation of the determined difference. Additionally or alternatively, as indicated in block 444, the wearable compute device 100 may convey a thermal signal, such as increase in temperature in an area where the wearable compute device 100 is in physical contact with the wearer. The wearable compute device 100 may, in some embodiments, decrease in temperature as the determined difference between the wearer's physiological functions and the reference physiological functions decreases, and increase in temperature as the determined difference increases, or vice versa.

In block 446, the wearable compute device 100 determines whether the compared physiological functions of the wearer and the person associated with the reference physiological rate data are synchronized, based on the determined difference. In doing so, as indicated in block 448, the illustrative wearable compute device 100 compares the determined difference to a threshold difference. For example, the determined difference may be a numeric value indicative of a difference in frequency, phase, and/or amplitude between compared heart rate data, respiration rate data, or heart rate variability data and the threshold difference may be a numeric value indicative of an acceptable level of synchronization. It should be appreciated that the threshold difference may be greater than zero, meaning the compared physiological rates need not be identical in order to be determined to be synchronized by the wearable compute device 100.

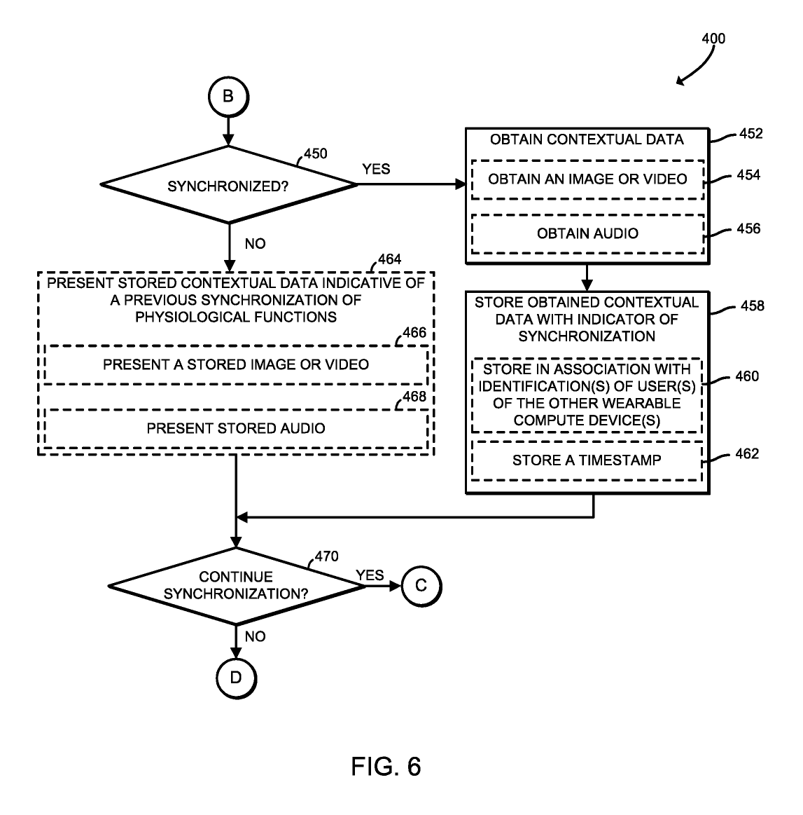

After the wearable compute device 100 determines whether the compared physiological functions of the wearer and the person associated with the reference physiological rate data are synchronized in block 446, the method 400 advances to block 450 of FIG. 6. In block 450, the wearable compute device 100 determines whether the compared physiological rates of the wearer are synchronized with those of the one or more other wearers. If so, the method advances to block 452, in which the wearable compute device 100 obtains contextual data. In doing so, as indicated in block 454, the wearable compute device 100 may obtain an image or video, such as an image or video of the wearer and a person or people with whom the wearer is synchronized. As indicated in block 456, the wearable compute device 100 may obtain audio, such a recording of environmental sounds, a conversation, or other audio indicative of the present circumstances of the wearer. In other words, the wearable compute device 100 obtains data regarding the surrounding circumstances of the wearer that are associated with the physiological functions being synchronized with the other wearer(s). In block 458, the wearable compute device 100 stores the obtained contextual data with an indicator that the physiological functions were synchronized at that time. As indicated in block 460, the wearable compute device 100 may store the obtained contextual data in association with an identification of the person or people with whom the wearer's physiological functions were synchronized. Also, as indicated in block 462, the wearable compute device 100 may store a timestamp of the time associated with the obtained contextual data.

If, in block 450, the wearable compute device 100 instead determines that the physiological functions are not synchronized, the illustrative wearable compute device 100 presents stored contextual data indicative of a previous synchronization of the physiological functions. In doing so, the wearable compute device 100 may presented a stored image or video clip, as indicated in block 466. As indicated in block 468, the wearable compute device 100 may additionally or alternatively present stored audio. In the illustrative embodiment, the wearable compute device 100 selects stored contextual data that is associated with the one or more people with whom the wearer is to be synchronized (i.e., the wearer(s) of the wearable compute device(s) that are in communication with the present wearable compute device 100). In response to receiving the presented contextual data from a previous time when the wearer was synchronized with the one or more other people, the wearer may be physiologically prompted to adjust his or her physiological function rates to be more synchronized with those of the one or more other people. Of course, if the wearable compute device 100 does not presently have any previously stored contextual data associated with a time when the physiological functions were synchronized, the wearable compute device 100 will not present any such contextual data. In block 470, the wearable compute device 100 determines whether to continue synchronization. In the illustrative embodiment, the wearable compute device 100 may continually operate to achieve and maintain synchronization unless the wearable compute device 100 receives a request to stop, such as through a user interface or from another wearable compute device 100, after a predefined time limit elapses, or after any other condition associated with discontinuing synchronization has occurred. If the wearable compute device 100 determines to continue operating to achieve and/or maintain synchronization, the method 400 loops back to block 410 of FIG. 4, in which the wearable compute device 100 again determines the physiological rate data 302 of the wearer of the wearable compute device 100. Otherwise, the method 400 loops back to block 402 of FIG. 4, in which the wearable compute device 100 determines whether to synchronize physiological functions.

### EXAMPLES

Illustrative examples of the technologies disclosed herein are provided below. An embodiment of the technologies may include any one or more, and any combination of, the examples described below.

Example 1 includes a wearable compute device for synchronizing physiological functions of a user with reference physiological rate data, the wearable compute device comprising at least one physiological sensor to produce sensor data indicative of one or more physiological functions of the user; at least one signal conveyor device; a physiological sensor manager module to determine physiological rate data of the user based on the sensor data produced by the at least one physiological sensor; a physiological rate comparison module to determine a difference between the determined physiological rate data of the user and the reference physiological rate data; and a signal generator module to generate a perceptible signal that is representative of the determined difference and convey the perceptible signal to the user with the at least one signal conveyor device to facilitate synchronization of the physiological functions of the user with the reference physiological rate data.

Example 2 includes the subject matter of Example 1, and further including a network communication module to receive the reference physiological rate data from a second wearable compute device associated with at least one other person.

Example 3 includes the subject matter of any of Examples 1 and 2, and further including a network communication module to transmit the detected physiological rate data to a second wearable compute device worn by another person.

Example 4 includes the subject matter of any of Examples 1-3, and wherein the physiological rate comparison module is further to identify an anomaly in the detected physiological rate data; and remove the anomaly from the detected physiological rate data before the determination of the difference between the detected physiological rate data and the reference physiological rate data.

Example 5 includes the subject matter of any of Examples 1-4, and wherein to identify the anomaly comprises to identify a change in the detected physiological rate data; determine whether the change satisfies a predefined threshold; and identify, in response to a determination that the change satisfies the predefined threshold, the change as an anomaly.

Example 6 includes the subject matter of any of Examples 1-5, and wherein to detect the physiological rate data comprises to detect at least one of a heart rate, a respiration rate, or a heart rate variability.

Example 7 includes the subject matter of any of Examples 1-6, and wherein the physiological rate comparison module is further to determine that the difference satisfies a predefined threshold and the wearable compute device further comprises at least one context sensor to produce sensor data; and a context manager module to determine contextual data indicative of a present context of the user and another person based on the sensor data produced by the context sensor, and store the obtained contextual data.

Example 8 includes the subject matter of any of Examples 1-7, and wherein the physiological rate comparison module is further to determine that the difference satisfies a predefined threshold and the wearable compute device further comprises a context manager module to present contextual data to the user indicative of an identified time when the physiological functions of the user were determined to be synchronized with physiological functions of another person.

Example 9 includes the subject matter of any of Examples 1-8, and wherein to present the contextual data comprises to present an image of a context of the user and the other person associated with the identified time when the physiological functions of the user were determined to be synchronized with the physiological functions of the other person.

Example 10 includes the subject matter of any of Examples 1-9, and wherein to convey the perceptible signal comprises to convey at least one of a haptic signal, a visual signal, an audible signal, or a thermal signal.

Example 11 includes the subject matter of any of Examples 1-10, and wherein to generate the perceptible signal comprises to determine an intensity of the perceptible signal based on a magnitude of the determined difference.

Example 12 includes the subject matter of any of Examples 1-11, and wherein to generate the perceptible signal comprises to determine a frequency of the perceptible signal based on a magnitude of the determined difference.

Example 13 includes the subject matter of any of Examples 1-12, and further including a network communication module to pair with a second wearable compute device associated with another person; and receive the reference physiological rate data from the second wearable compute device after the wearable compute device has paired with the second wearable compute device.

Example 14 includes the subject matter of any of Examples 1-13, and wherein the signal generator module is further to convey a haptic signal representative of the reference physiological rate data to the user.

Example 15 includes the subject matter of any of Examples 1-14, and further including a network communication module to determine a transmission latency associated with the reference physiological rate data, and the physiological rate comparison module is further to adjust the reference physiological rate data based on the determined transmission latency.

Example 16 includes a method for synchronizing physiological functions of a user with reference physiological rate data, comprising determining, by the wearable compute device, physiological rate data of the user of the wearable compute device based on sensor data produced by at least one physiological sensor included in the wearable compute device, wherein the sensor data is indicative of one or more physiological functions of the user; determining, by the wearable compute device, a difference between the determined physiological rate data of the user and the reference physiological rate data; generating, by the wearable compute device, a perceptible signal that is representative of the determined difference; and conveying, by the wearable compute device, the perceptible signal to the user with at least one signal conveyor device included in the wearable compute device, to facilitate synchronization of the physiological functions of the user with the reference physiological rate data.

Example 17 includes the subject matter of Example 16, and further including receiving, by the wearable compute device, the reference physiological rate data from a second wearable compute device associated with at least one other person.

Example 18 includes the subject matter of any of Examples 16 and 17, and further including transmitting, by the wearable compute device, the detected physiological rate data to a second wearable compute device worn by another person.

Example 19 includes the subject matter of any of Examples 16-18, and further including identifying, by the wearable compute device, an anomaly in the detected physiological rate data; and removing, by the wearable compute device, the anomaly from the detected physiological rate data before the determination of the difference between the detected physiological rate data and the reference physiological rate data.

Example 20 includes the subject matter of any of Examples 16-19, and wherein identifying the anomaly comprises identifying a change in the detected physiological rate data; determining whether the change satisfies a predefined threshold; and identifying, in response to a determination that the change satisfies the predefined threshold, the change as an anomaly.

Example 21 includes the subject matter of any of Examples 16-20, and wherein detecting the physiological rate data comprises detecting at least one of a heart rate, a respiration rate, or a heart rate variability.

Example 22 includes the subject matter of any of Examples 16-21, and wherein the wearable compute device includes a context sensor to produce sensor data, the method further comprising determining, by the wearable compute device, that the difference satisfies a predefined threshold; determining, by the wearable compute device, contextual data indicative of a present context of the user and another person based on the sensor data produced by the context sensor; and storing, by the wearable compute device, the obtained contextual data.

Example 23 includes the subject matter of any of Examples 16-22, and further including determining, by the wearable compute device, that the difference satisfies a predefined threshold; and presenting, by the wearable compute device, contextual data to the user indicative of an identified time when the physiological functions of the user were determined to be synchronized with physiological functions of another person.

Example 24 includes the subject matter of any of Examples 16-23, and wherein presenting the contextual data comprises presenting an image of a context of the user and the other person associated with the identified time when the physiological functions of the user were determined to be synchronized with the physiological functions of the other person.

Example 25 includes the subject matter of any of Examples 16-24, and wherein conveying the perceptible signal comprises conveying at least one of a haptic signal, a visual signal, an audible signal, or a thermal signal.

Example 26 includes the subject matter of any of Examples 16-25, and wherein generating the perceptible signal comprises determining an intensity of the perceptible signal based on a magnitude of the determined difference.

Example 27 includes the subject matter of any of Examples 16-26, and wherein generating the perceptible signal comprises determining a frequency of the perceptible signal based on a magnitude of the determined difference.

Example 28 includes the subject matter of any of Examples 16-27, and further including pairing the wearable compute device with a second wearable compute device associated with another person; and receiving the reference physiological rate data from the second wearable compute device after pairing with the wearable compute device with the second wearable compute device.

Example 29 includes the subject matter of any of Examples 16-28, and further including conveying a haptic signal representative of the reference physiological rate data to the user.

Example 30 includes the subject matter of any of Examples 16-29, and further including determining, by the wearable compute device, a transmission latency associated with the reference physiological rate data; and adjusting, by the wearable compute device, the reference physiological rate data based on the determined transmission latency.

Example 31 includes one or more computer-readable storage media comprising a plurality of instructions that, when executed by a wearable compute device, cause the wearable compute device to perform the method of any of Examples 16-30.

Example 32 includes a wearable compute device for synchronizing physiological functions of a user with reference physiological rate data, comprising means for determining physiological rate data of the user of the wearable compute device based on sensor data produced by at least one physiological sensor included in the wearable compute device, wherein the sensor data is indicative of one or more physiological functions of the user; means for determining a difference between the determined physiological rate data of the user and the reference physiological rate data; means for generating a perceptible signal that is representative of the determined difference; and means for conveying the perceptible signal to the user with at least one signal conveyor device included in the wearable compute device, to facilitate synchronization of the physiological functions of the user with the reference physiological rate data.

Example 33 includes the subject matter of Example 32, and, further including means for receiving the reference physiological rate data from a second wearable compute device associated with at least one other person.

Example 34 includes the subject matter of any of Examples 32 and 33, and further including means for transmitting the detected physiological rate data to a second wearable compute device worn by another person.

Example 35 includes the subject matter of any of Examples 32-34, and further including means for identifying an anomaly in the detected physiological rate data; and means for removing the anomaly from the detected physiological rate data before the determination of the difference between the detected physiological rate data and the reference physiological rate data.

Example 36 includes the subject matter of any of Examples 32-35, and wherein identifying the anomaly comprises means for identifying a change in the detected physiological rate data; means for determining whether the change satisfies a predefined threshold; and means for identifying, in response to a determination that the change satisfies the predefined threshold, the change as an anomaly.

Example 37 includes the subject matter of any of Examples 32-36, and wherein the means for detecting the physiological rate data comprises means for detecting at least one of a heart rate, a respiration rate, or a heart rate variability.

Example 38 includes the subject matter of any of Examples 32-37, and further including means for determining that the difference satisfies a predefined threshold; means for determining contextual data indicative of a present context of the user and another person based on sensor data produced by a context sensor; and means for storing the obtained contextual data.

Example 39 includes the subject matter of any of Examples 32-38, and further including means for determining that the difference satisfies a predefined threshold; and means for presenting contextual data to the user indicative of an identified time when the physiological functions of the user were determined to be synchronized with physiological functions of another person.

Example 40 includes the subject matter of any of Examples 32-39, and wherein the means for presenting the contextual data comprises means for presenting an image of a context of the user and the other person associated with the identified time when the physiological functions of the user were determined to be synchronized with the physiological functions of the other person.

Example 41 includes the subject matter of any of Examples 32-40, and wherein the means for conveying the perceptible signal comprises means for conveying at least one of a haptic signal, a visual signal, an audible signal, or a thermal signal.

Example 42 includes the subject matter of any of Examples 32-41, and wherein the means for generating the perceptible signal comprises means for determining an intensity of the perceptible signal based on a magnitude of the determined difference.

Example 43 includes the subject matter of any of Examples 32-42, and wherein the means for generating the perceptible signal comprises means for determining a frequency of the perceptible signal based on a magnitude of the determined difference.

Example 44 includes the subject matter of any of Examples 32-43, and further including means for pairing the wearable compute device with a second wearable compute device associated with another person; and means receiving the reference physiological rate data from the second wearable compute device after pairing with the wearable compute device with the second wearable compute device.

Example 45 includes the subject matter of any of Examples 32-44, and further including means for conveying a haptic signal representative of the reference physiological rate data to the user.

Example 46 includes the subject matter of any of Examples 32-45, and further including means for determining a transmission latency associated with the reference physiological rate data; and means for adjusting the reference physiological rate data based on the determined transmission latency.

1. A wearable compute device for synchronizing physiological functions of a user with reference physiological rate data, the wearable compute device comprising:
at least one physiological sensor to produce sensor data indicative of one or more physiological functions of the user;
at least one signal conveyor device;
a physiological sensor manager module to determine physiological rate data of the user based on the sensor data produced by the at least one physiological sensor;
a physiological rate comparison module to determine a difference between the determined physiological rate data of the user and the reference physiological rate data; and
a signal generator module to generate a perceptible signal that is representative of the determined difference and convey the perceptible signal to the user with the at least one signal conveyor device to facilitate synchronization of the physiological functions of the user with the reference physiological rate data.

2. The wearable compute device of claim 1, further comprising a network communication module to receive the reference physiological rate data from a second wearable compute device associated with at least one other person.

3. The wearable compute device of claim 1, further comprising a network communication module to transmit the detected physiological rate data to a second wearable compute device worn by another person.

4. The wearable compute device of claim 1, wherein the physiological rate comparison module is further to:
identify an anomaly in the detected physiological rate data; and
remove the anomaly from the detected physiological rate data before the determination of the difference between the detected physiological rate data and the reference physiological rate data.

5. The wearable compute device of claim 4, wherein to identify the anomaly comprises to:
identify a change in the detected physiological rate data;
determine whether the change satisfies a predefined threshold; and
identify, in response to a determination that the change satisfies the predefined threshold, the change as an anomaly.

6. The wearable compute device of claim 1, wherein to detect the physiological rate data comprises to detect at least one of a heart rate, a respiration rate, or a heart rate variability.

7. The wearable compute device of claim 1, wherein the physiological rate comparison module is further to determine that the difference satisfies a predefined threshold and the wearable compute device further comprises:
at least one context sensor to produce sensor data; and
a context manager module to determine contextual data indicative of a present context of the user and another person based on the sensor data produced by the context sensor, and store the obtained contextual data.

8. The wearable compute device of claim 1, wherein the physiological rate comparison module is further to determine that the difference satisfies a predefined threshold and the wearable compute device further comprises:
a context manager module to present contextual data to the user indicative of an identified time when the physiological functions of the user were determined to be synchronized with physiological functions of another person.

9. The wearable compute device of claim 8, wherein to present the contextual data comprises to present an image of a context of the user and the other person associated with the identified time when the physiological functions of the user were determined to be synchronized with the physiological functions of the other person.

10. One or more computer-readable storage media comprising a plurality of instructions that, when executed by a wearable compute device, cause the wearable compute device to:
determine physiological rate data of a user of the wearable compute device based on sensor data produced by at least one physiological sensor included in the wearable compute device, wherein the sensor data is indicative of one or more physiological functions of the user;
determine a difference between the determined physiological rate data of the user and reference physiological rate data;
generate a perceptible signal that is representative of the determined difference; and
convey the perceptible signal to the user with at least one signal conveyor device included in the wearable compute device, to facilitate synchronization of the physiological functions of the user with the reference physiological rate data.

11. The one or more computer-readable storage media of claim 10, wherein the plurality of instructions further cause the wearable compute device to receive the reference physiological rate data from a second wearable compute device associated with at least one other person.

12. The one or more computer-readable storage media of claim 10, wherein the plurality of instructions further cause the wearable compute device to transmit the detected physiological rate data to a second wearable compute device worn by another person.

13. The one or more computer-readable storage media of claim 10, wherein the plurality of instructions further cause the wearable compute device to:
identify an anomaly in the detected physiological rate data; and
remove the anomaly from the detected physiological rate data before the determination of the difference between the detected physiological rate data and the reference physiological rate data.

14. The one or more computer-readable storage media of claim 13, wherein to identify the anomaly comprises to:
identify a change in the detected physiological rate data;
determine whether the change satisfies a predefined threshold; and
identify, in response to a determination that the change satisfies the predefined threshold, the change as an anomaly.

15. The one or more computer-readable storage media of claim 10, wherein to detect the physiological rate data comprises to detect at least one of a heart rate, a respiration rate, or a heart rate variability.

16. The one or more computer-readable storage media of claim 10, wherein the wearable compute device includes a context sensor to produce sensor data and the plurality of instructions further cause the wearable compute device to:
determine that the difference satisfies a predefined threshold;
determine contextual data indicative of a present context of the user and another person based on the sensor data produced by the context sensor; and
store the obtained contextual data.

17. The one or more computer-readable storage media of claim 10, wherein the plurality of instructions further cause the wearable compute device to:
determine that the difference satisfies a predefined threshold; and
present contextual data to the user indicative of an identified time when the physiological functions of the user were determined to be synchronized with physiological functions of another person.

18. A method for synchronizing physiological functions of a user with reference physiological rate data, comprising:
determining, by the wearable compute device, physiological rate data of the user of the wearable compute device based on sensor data produced by at least one physiological sensor included in the wearable compute device, wherein the sensor data is indicative of one or more physiological functions of the user;
determining, by the wearable compute device, a difference between the determined physiological rate data of the user and the reference physiological rate data;
generating, by the wearable compute device, a perceptible signal that is representative of the determined difference; and
conveying, by the wearable compute device, the perceptible signal to the user with at least one signal conveyor device included in the wearable compute device, to facilitate synchronization of the physiological functions of the user with the reference physiological rate data.

19. The method of claim 18, further comprising receiving, by the wearable compute device, the reference physiological rate data from a second wearable compute device associated with at least one other person.

20. The method of claim 18, further comprising transmitting, by the wearable compute device, the detected physiological rate data to a second wearable compute device worn by another person.

21. The method of claim 18, further comprising:
identifying, by the wearable compute device, an anomaly in the detected physiological rate data; and
removing, by the wearable compute device, the anomaly from the detected physiological rate data before the determination of the difference between the detected physiological rate data and the reference physiological rate data.

22. The method of claim 21, wherein identifying the anomaly comprises:
identifying a change in the detected physiological rate data;
determining whether the change satisfies a predefined threshold; and
identifying, in response to a determination that the change satisfies the predefined threshold, the change as an anomaly.

23. The method of claim 18, wherein detecting the physiological rate data comprises detecting at least one of a heart rate, a respiration rate, or a heart rate variability.

24. The method of claim 18, wherein the wearable compute device includes a context sensor to produce sensor data, the method further comprising:
determining, by the wearable compute device, that the difference satisfies a predefined threshold;
determining, by the wearable compute device, contextual data indicative of a present context of the user and another person based on the sensor data produced by the context sensor; and
storing, by the wearable compute device, the obtained contextual data.

25. The method of claim 18, further comprising:
determining, by the wearable compute device, that the difference satisfies a predefined threshold; and
presenting, by the wearable compute device, contextual data to the user indicative of an identified time when the physiological functions of the user were determined to be synchronized with physiological functions of another person.
